(19) United States
(12) Patent Application Publication
(10) Pub. No.: US 2008/0171072 A1
(43) Pub. Date: Jul. 17, 2008

(54) OCULAR INSERTS CONTAINING APOMORPHINE

(76) Inventors: Frank Burczynski, Manitoba (CA); Dennis Cote, Manitoba (CA)

Correspondence Address: ADE & COMPANY INC., 2157 Henderson Highway, WINNIPEG, MB, R2G1P9, omitted

(21) Appl. No.: 11/836,232

(22) Filed: Aug. 9, 2007

Related U.S. Application Data
(60) Provisional application No. 60/836,951, filed on Aug. 11, 2006.

(51) Int. Cl. A61F 9/00; A61K 31/439; A61P 27/02

(52) U.S. Cl. 424/427; 514/289

(57) ABSTRACT

A gelatin-based insert was designed to deliver apomorphine by the ocular route. A clinical trial showed the product to have an efficacy similar to intravenously-administered apomorphine with a better safety profile in terms of adverse effects.

### PRIOR APPLICATION INFORMATION

This application claims the benefit of U.S. Provisional Patent Application 60/836,951, filed Aug. 11, 2006.

### BACKGROUND OF THE INVENTION

Ocular inserts have a number of potential advantages over other dose forms as a system for delivering medication either locally to the eye or systemically using the eye as an entry point. Bonferoni et al (Bonferoni et al., 2004, Eur J Pharm Biopharm 57: 465-472) discussed precorneal loss due to lacrimal flow and blinking and demonstrated that these problems could be reduced through the use of a solid carageenan-gelatin delivery system and Friedrich et al (Friedrich et al., 1996, J Ocul Pharmacol Ther 12: 5-18) discussed the pharmacokinetic difference between ocular inserts and eye drops and showed significantly improved bioavailability with solid ocular inserts. A number of ocular inserts have been described and these include inserts containing cellulose derivatives for treatment of ‘dry eye’ or keratoconjunctivitis sicca (LaMotte et al., 1985, J Am Optom Assoc 56: 298-302; Gelatt et al., 1979, Am J Vet Res 40: 702-704), inserts for delivery of local anesthetics (Mahe et al., 2005, Br J Clin Pharmacol 59: 200-226) and inserts for prolonged release of antibiotics (Baeyens et al., 2002, J Control Release 85: 163-168; Sultana et al., 2005, Acta Pharma 55: 305-314; Baeyens et al., 1998, J Control Release 52: 215-220; Dicolo et al., 2001, Int J Pharm 215: 101-111; Gurtler et al., 1995, Pharm Res 12: 1791-1795; Hosaka et al., 1983, Biomaterials 4: 243-248). Gurtler and Gurny (Gurtler and Gurny, 1995, Drug Dev Ind Pharm 21: 1-18) defined an ophthalmic insert as being a sterile product with a solid or semi-solid consistency in a size and shape suitable for ocular application. These inserts can be used for topical or systemic therapy and the purpose of this dose form is to increase the contact time between the device and the ocular issues to ensure a sustained release and subsequent therapeutic effect. The devices designed to produce systemic effect usually used absorbable gelatin as a carrier. The authors divided the ocular inserts into two general groups: insoluble and soluble and in the insoluble groups described diffusional, osmotic and contact lens systems. A diffusional system consisted of a central reservoir of drug enclosed by a semipermeable membrane where the solvent system in the reservoir was glycerin, propylene glycol or an oil mixture and the membrane was composed of polycarbonate, polyvinyl or polyamine derivatives. Osmotic systems were composed of a central part with two components; the drug is surrounded by polymer dispersed in an osmotic solute and the entire device enclosed by a semi-permeable membrane. The matrix polymer is usually based on an ethylene vinyl ester, the osmotic solute may be sodium chloride, calcium lactate or a phosphate salt and the enclosing membrane is usually composed of a cellulose acetate derivative although little information on the composition of the contact lens systems was presented as these are usually proprietary. Drug loading is achieved through soaking the device in a solution of the drug followed by a drying process. The soluble group consisted of inserts made from natural materials or synthetic polymers. The natural materials such as collagen were loaded with drug again through a soaking/drying process. Synthetic polymers used are generally cellulose derivatives containing plasticizers such as polyethylene glycol, propylene glycol or glycerin and these may be coated with an enteric polymer such as cellulose acetate phthalate. The last in the soluble group is described as bioerodible and the matrix material is usually cross-linked gelatin or polyester or polycarbonate derivatives.

### SUMMARY OF THE INVENTION

According to a first aspect of the invention, there is provided a removable ocular insert comprising an effective amount of apomorphine in a polymer matrix.

According to a second aspect of the invention, there is provided a method of inducing emesis in an animal in need of such treatment comprising inserting into the eye of said animal a removable ocular insert comprising an effective amount of apomorphine in a polymer matrix, and once emesis has occurred, removing the insert from the eye of said animal.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Described herein is a method of manufacturing ocular inserts comprising an effective amount of apomorphine and a method of use thereof for inducing emesis.

As discussed below, the ocular inserts are arranged for insertion into the eye and comprise an effective amount of apomorphine. As discussed below, as used herein, ‘an effective amount’ is an amount of apomorphine that is sufficient to induce emesis in a reasonable amount of time. As discussed below, in some embodiments, ‘a reasonable amount of time’ is 30 minutes or less or may be 15 minutes or less. However, as will be appreciated by one of skill in the art, this will depend on the formulation used, the reason for which induction of emesis is needed (potential severity of condition) as well as other factors known to one of skill in the art.

As will be appreciated by one of skill in the art and as discussed herein, the instant invention provides a method for delivering a controlled amount of apomorphine in a manner in which administration of the apomorphine can be stopped by removing the insert. This represents a considerable improvement over for example injection of apomorphine because the quantity of apomorphine required to induce emesis cannot always be predicted in advance, for example, based on body weight. As discussed herein, if too little is administered, emesis is not induced and a second dosage may be ineffective. If too large a dosage is administered, emesis is induced but may be uncontrollable and may lead to other side-effects as discussed below. However, by administering apomorphine in a polymer matrix in the form of removable ocular inserts, the inserts can be removed once emesis occurs, thereby limiting side effects as discussed below.

In one embodiment of the invention, the ocular insert comprises an effective amount of apomorphine admixed with a suitable polymer and a plasticizer. In some embodiments, the insert may include at least one antioxidant.

The apomorphine may be apomorphine HCI or another suitable form of apomorphine, for example, a pharmaceutically acceptable salt of apomorphine. In a preferred embodiment, the ocular insert comprises 0.5 mg to 5 mg apomorphine, for example, apomorphine HCI. As discussed below, in a preferred embodiment, the insert comprises about 1.0 to about 5.0 mg apomorphine. In a further preferred embodiment, the insert comprises about 1.0 to about 3.0 mg apomorphine. In a yet further preferred embodiment, the insert comprises about 2.0 mg apomorphine.

In a preferred embodiment, the polymer is gelatin.

In a preferred embodiment, the plasticizer is a polyol. More preferably, the polyol is not a polyol sugar. In some embodiments, the plasticizer is selected from the group consisting of glycerin, propylene glycol and polyethylene glycol.

In a yet further preferred embodiment, glycerin is mixed with gelatin at a ratio between 1:1 to 1:2.5, as discussed below, or at a ratio of 1:1 to 1:1.5.

As discussed below, other suitable biocompatible polymer/plasticizer combinations having the desired characteristics of tackiness, suitable drug release profile, stability, sterility and integrity (for ease of removal) may be used.

In some embodiments, the antioxidant is a mixture of metabisulfite and ascorbic acid. As will be appreciated by one of skill in the art, other suitable antioxidants which prevent oxidation of apomorphine and are substantially biocompatible may be used in the ocular inserts as described herein.

As discussed below, the apomorphine is mixed with the polymer and plasticizer and the mixture is allowed to dry into a film.

In a preferred embodiment, the film has a thickness of about 0.8 mm to about 1.2 mm, for example, 1 mm. As discussed herein, in some embodiments, the mixture includes at least one antioxidant so that the film is substantially transparent.

As discussed herein, in some embodiments, the insert is cut from the film such that the insert has a diameter of about 6 mm.

As discussed below, the removable ocular inserts are used to induce emesis in a patient in need of such treatment, for example, a patient that is in need of having emesis inducted, for example, a patient that has or is suspected of having an ingesting a foreign or undesirable material. Examples of such foreign or undesirable material will be readily apparent to one of skill in the art and examples of same are provided below.

In use, the ocular insert comprising an effective amount of apomorphine is inserted into the eye of an animal in need of such treatment. Once emesis is induced, the insert may be removed.

In some embodiments, inserts may be placed in both eyes of the patient.

In a preferred embodiment, the patient is an animal. In a yet further preferred embodiment, the patient is a non-human animal. Preferably, the animal is a non-human domestic animal. More preferably, the animal is a canine.

Rapid but controlled release is desired and a matrix type of delivery system would be appropriate. Only two reports could be found where apomorphine was delivered through a matrix system; in one report, Ugwoke et al (Ugwoke et al., 1997, Int J Pharm 148: 23-32) formed gelatin microspheres containing apomorphine to be studied in the treatment of Parkinsonism and in the second, Raasch et al (Raash et al., 2000, Jpn J Pharmacol 84: 36-43) developed and conducted release studies, both in vitro and in vivo using rats, of an insert containing apomorphine in an ethylene vinyl acetate polymer which was also to be used in the treatment of Parkinson's disease. Both of these systems were designed for long-term release of apomorphine with the microsphere system to be used as for nasal administration while the system by Raasch et al was designed as a subcutaneous implant system.

The insert is arranged to be non-irritating to the conjunctival membranes; be easy to apply and soft and adhesive enough to remain in place; release most of the drug within a five to ten minute time window; have a robust matrix to allow for easy removal when emesis has occurred; have sufficient chemical stability to allow a reasonable shelf life; and the insert must be able to be sterilized.

The matrix combination must allow for rapid but controlled release while maintaining sufficient product integrity to allow for easy removal from the eye after the therapeutic end-point has been reached. Apomorphine may require additional substances for example an antioxidant or buffer system but the total solute load of the insert will be limited since ocular irritation will be a factor; mild irritation will be necessary to ensure adequate tear flow to provide solvent to allow drug release but excessive tearing will result in the drug being washed away before absorption can occur.

Gelatin, polyvinylpyrolidone (PVP) and hydroxypropylmethylcellulose (HPMC) were selected as candidate polymers and glycerin and triethyl citrate were selected as plasticizers. In one embodiment, a circular insert about 6 mm in diameter and 1 mm in thickness would be an appropriate size for insertion into the conjunctival sac and the method of preparation would be casting of a solution to form a film. The dimensions provide an insert which was large enough and suitably robust for the clinician to handle and place into the eye yet small enough to fit easily into the eye.

The emetic dose selected for apomorphine in canines was 0.1 mg/kg (Scherkl et al., 1990, J Vet Pharmacol Therap 13: 154-158) so the drug load per insert was set at 2 mg which would provide the appropriate dose for a 20 kg canine patient. Since the drug release will be controlled, this drug load would be appropriate for patients with weights ranging from 5 to 20 kg although larger patients may require an insert in each eye and very small patients may require only half of an insert.

Preliminary screening of polymer-plasticizer combinations suggested that films could be formed using one part of glycerin to one and one half parts polymer and one part of triethyl citrate to nine parts of polymer so these ratios were used as the mid-point and three levels of concentration were examined for each polymer-plasticizer combination. The matrix formulations evaluated are presented in Table 1.

Since all of the materials used were water soluble, water was used as the solvent with a total solute load of 5 g. The solutions were cast in 100-mm Petri dishes and the films were formed and cured by placing them in a level class ‘A’ biological containment cabinet with a vertical air flow of 24 m/min. A film was usually formed after 24 hours at which time the film could be pulled from the Petri dish and cured for 48 hours in a container over anhydrous silica gel. Inserts of 6-mm diameter could be cut using a circular punch and these could then be evaluated for suitability.

The films and discs resulting from the experiment were evaluated by grading the attributes of flexibility, clarity, tackiness and integrity after soaking in water (35° C.) for 10 minutes. In terms of flexibility, the ideal film would be flexible enough to conform to the curvature of the eye but rigid enough to be handled and easily inserted into the conjunctival sac. This attribute was graded with a score from 1-5 with the higher score being given for a good balance between rigidity and flexibility. The attribute of clarity was considered to be of lesser importance and was graded with a score from 1-3. Clarity in the film was desirable from an esthetic perspective but also would allow detection if one of the components of the product came out of solution during the casting or curing process which could lead to problems with content uniformity. Tackiness was another attribute where balance was important and it was graded with a score from 1-5. The film required sufficient tackiness to adhere to the eye tissue but not so tacky as to adhere to the surface of the packaging material and be difficult to use. Integrity after soaking in water was considered very important since one of the goals of the delivery system was to allow removal of the drug reservoir after the therapeutic end point of emesis was achieved. In order to allow easy removal at this point, it would be important that the device was intact and still contained what would be excess drug for the treatment. This attribute was scored from 1-6.

As shown in Table 2, the subjective overall assessment was that formulations 1 to 3 were somewhat brittle and opaque; formulations 4 to 6 gave acceptable films; formulations 7 to 9 were somewhat brittle; formulations 10 to 12 were quite tacky; formulations 13 to 15 were quite brittle and formulations 16 to 18 gave acceptable films.

The results of the MLR analysis are summarized in Tables 3 and 4 and from these data it can be seen that the selection of plasticizer (b2) in this model is very important with ethyl citrate having a negative effect on the overall score of the formulation. The interaction between the polymer and the plasticizer (b12) is also important with the combination of gelatin and glycerin having the most positive effect. The level of plasticizer present (b3) within the limits examined does not appear to be an important factor nor do the interactions between polymer, plasticizer and plasticizer level (b123).

Based on these data, the combination of gelatin and glycerin provide the closest fit to the desired insert attributes and although not a significant factor within the range studied, glycerin at an intermediate concentration was used. However, as discussed above, other suitable concentrations may be used depending of course on the desired characteristics of the insert.

Two different types of gelatin can be produced depending on the method used to pretreat the collagen; pretreatment with alkali hydrolyses the amide groups of asparagine and glutamine producing free carboxylic acid functional groups whereas pretreatment with acid does not (Young et al., 2005, J Control Release 109: 256-274). The result is that alkali-treated collagen yields gelatin with a larger number of free carboxylic acid groups making it more negatively charged and lowering the isoelectric point compared to acid treated collagen. This allows for flexibility in terms of enabling polygon complexation of the gelatin matrix with either positively or negatively charged drugs; acidic gelatin should be used for basic proteins or drugs while basic gelatin should be used for acidic agents (Bowman and Ofner, 2000, Pharm Res 17: 1309-1315). Either type A or type B gelatin is acceptable in the instant invention.

In terms of a preliminary formulation, the following was investigated further:

| | |
|---|---|
| Apomorphine HCl USP | 0.46 g |
| Gelatin NF | 2.80 g |
| Glycerin USP | 2.05 g |

The mixture was dissolved in a suitable aqueous medium and cast in 100 mm sterile Petrie dish. After curing each 10 mm circular insert carries a drug load of 2 mg.

For the initial casting of inserts using the gelatin and glycerin matrix with apomorphine included, the gelatin and glycerin were weighed into a 100-mL beaker, dispersed with about 35 mL of water and gently heated until the gelatin dissolved. The apomorphine was weighed and dissolved in about 15 mL of water with the aid of gentle heating and when dissolution was complete, this solution was added to the gelatin and glycerin, gently mixed avoiding air entrainment and the whole poured into a 100-mm disposable Petri dish. This mixture was placed in a level class ‘A’ biological containment cabinet (Baker) with a vertical air flow of about 24 m/min and allowed to cure, It was noted that the film formed had a distinct green discoloration suggesting that some decomposition of the apomorphine had occurred during the casting and curing process.

Apomorphine is subject to oxidative degradation to form an inactive quinone (Linde and Ragab, 1968, Helvetica Chimica Acta 51: 683-687). A number of formulation strategies are used to protect drugs from this type of degradation and include protection from exposure to light, excluding oxygen from the final packaging, including antioxidants in the formulation and formulating the product at an acidic pH (Waterman et al., 2002, Pharm Dev Tech 7: 1-32). Since ascorbic acid and metabisulfate or sodium sulfite had been shown to function as effective antioxidants for products containing apomorphine and both of these substances have been used in ophthalmic products, these were assessed as potential added substances to extend the shelf-life of the product (Lundgren and Landersjo, 1970, Acta Pharm Suec 7: 133-148). A number of authors have suggested that visible discoloration, usually green, of apomorphine may be present even when only 0.1% of the drug has decomposed (Burkman, 1965, J Pharm Sci 54: 325-326) and that the relationship between the intensity of discoloration and amount of drug decomposed is unclear (Lundgren and Landersjo, 1970; Kaul and Brochmann-Hanssen 1961, J Pharm Sci 50: 266-267; Burkman, 1963, J Pharm Pharmacol 15: 461-465).

The purpose of this experiment was to determine whether the addition of sodium metabisulfite and/or ascorbic acid would prevent discoloration of the inserts during the casting and curing process. The effect of the antioxidants on the degradation rate of apomorphine in the inserts at 80° C. was also investigated.

Four sets of apomorphine inserts were prepared by the casting process already described. One set of inserts were cast from a solution containing both ascorbic acid and sodium metabisulfite, one containing ascorbic acid alone, one containing sodium metabisulfite alone and one containing neither ascorbic acid or sodium metabisulfite. A concentration of 0.1% of the final casting solution volume was used for both agents; the formulations are presented in Table 5. For each formulation, samples consisting of four inserts were weighed and placed into 5-mL type 1 glass vials and polymeric closures applied. Four trials of each formulation were conducted. The vials were than placed into a water bath maintained at 80±0.1° C. and samples of each formulation were withdrawn at three day intervals and stored at −20° C. until analysis. After all the samples were collected, each was analyzed for apomorphine content using the developed HPLC method. Peak purity was monitored by comparison of the data obtained by UV detection to that obtained from the fluorescence and electrochemical detectors. Data analysis consisted of determining degradation apparent rate constants for each formulation.

Films cast with both ascorbic acid and sodium metabisulfite added to the apomorphine casting solution showed no discoloration while films cast from solutions where only ascorbic acid was added showed a pink discoloration and films cast with only metabisulfite added showed a faint blue-grey discoloration. The films cast from solutions with neither ascorbic acid nor sodium metabisulfite were distinctly green in color and from this it appeared that including sodium metabisulfite and ascorbic acid each in concentrations of 0.1% to the casting solution prevented the development of discoloration in the inserts during the casting and curing process.

The apomorphine present in the samples expressed as percentage of the labeled content (2 mg) is presented in Table 6 and these data were analyzed using regression analysis and assuming a first-order degradation process; apparent first-order rate constants and t₉₀ values were calculated from this analysis.

The findings of this experiment suggest that there is an interaction between bisulfite and apomorphine and in their studies of apomorphine stability, Lundgren and Landersjo (Lundgren and Landersjo, 1970, Acta Pharm Suec 7: 133-148) noted a rapid but slight decline in apomorphine concentration when it was mixed with bisulfite and heated. Subsequent examination of the solution using paper chromatography showed the presence of a yellow spot which had not been present before and they suggested that this spot, might be the product of a reaction between apomorphine and bisulfite.

The addition of sodium metabisulfite to the inserts appeared to result in the immediate loss of a small amount of apomorphine but this loss appeared to be reduced when bisulfite and ascorbic acid were used together. Since tears would be necessary to allow release of the drug, a low level irritation would be desirable whereas excessive tearing would cause the released drug to be washed away before drug absorption could take place. The residual ascorbic acid and sodium metabisulfite left in the inserts from the casting and curing process could cause minor irritation and tearing which would be desirable provided the tearing is not excessive. Since a preliminary trial of autoclaving the casting solution was unsuccessful because apomorphine is unstable under conditions required for autoclaving as the process caused severe discoloration of the solution, sterilization of the gelatin, glycerin and some of the water using autoclaving and sterilization of the apomorphine, ascorbic acid and sodium metabisulfite dissolved in the remainder of the water using membrane filtration was considered. The two sterile solutions could be aseptically mixed and cast into sterile Petri dishes then cured and dried in the sterile environment of a containment cabinet.

The appropriate amounts of glycerin and gelatin were dissolved with gentle heating in about 40 mL of water for injection and placed into a 50-mL type one glass vial and a polymeric closure affixed. The vial was then placed in an autoclave and steam-sterilized at 121° C. (15 psig) for 15 minutes. The appropriate amounts of apomorphine, sodium metabisulfite and ascorbic acid were dissolved with gentle heating in about 10 mL of water for injection and allowed to cool. After sterilization, the gelatin-glycerin solution was maintained at 45° C. to prevent setting and the apomorphine solution was drawn up into a 10 mL syringe and a sterile disposable membrane (0.2 gm) and sterile vented needle were affixed. The apomorphine solution was then sterile-filtered into the gelatin-glycerin solution and the solution remaining in the filter housing rinsed through with an additional 3 mL of water for injection. The solutions were then gently mixed, the vial closure removed and the solution was poured into a sterile Petri dish and allowed to set and cure. After 48 hours of curing, a satisfactory film had formed and this was removed from the Petri dish and inserts cut from the film using a punch which had been sterilized by autoclaving. The inserts were then packaged into the sterilized plastic wells and the sterilized label-backing applied. All the procedures were done aseptically using accepted standards of practice and all the procedures were done in a biological containment cabinet with a vertical air flow of 24 meters per minute.

After storage for a week under ambient conditions, five of the inserts were tested for sterility. This was done using sterile Trypticase soy broth and positive, negative and main positive controls were simultaneously run with the samples. The test organisms used in the positive controls were Staphylococcus aureus (ATCC 25923), Bacillus subtilis (ATCC 66333) and Pseudomonas aeruginosa (ATCC 27853).

The procedure outlined where two solutions were prepared and sterilized separately was workable and a sterile casting solution was obtained. The sterile casting solution was allowed to set and cure in the sterile environment of a biological containment cabinet. The cast film was removed from the Petri dish aseptically and inserts were cut using a sterile punch then the inserts were packaged into the sterile plastic blister wells and scaled with the sterilized labels.

Seven days after preparation, sterility tests were run on five of the inserts and none of the samples tested showed growth in Trypticase soy broth after seven days incubation at 35° C.; the positive controls showed growth and the negative controls showed no growth.

A suitable matrix for the inserts was developed and a base of gelatin and glycerin was selected with sodium metabisulfite and ascorbic acid added to prevent degradation of the apomorphine during the casting and drying process.

Historically emetic agents have been used to induce vomiting in cases of oral ingestion of poisons but their use for this purpose has declined dramatically over the past two decades due to the results of studies which have demonstrated that emesis lacks clinic efficacy as a therapeutic tool for humans in these cases. In veterinary medicine, however, there are situations where emesis is still indicated, particularly in canine patients. The nature of the intoxicants ingested by dogs include items such as garbage and carrion, the quantity of material ingested is often very large and with dogs the packaging materials holding the intoxicant are often consumed along with the intoxicant. In human medicine more than half of the accidental poisonings occur in children under the age of six years whereas with dogs there does not seem to be much of an age bias and the probability of exposure seems quite consistent over the life-span of a dog.

Apomorphine is a very potent emetic agent which has fallen into disuse. Apomorphine is a dopamine D2 agonist able to interact with central dopamine D2-receptors and acts at the level of the chemoreceptor trigger zone (CTZ) in the area postrema of the medulla (Lang et al., 1988, Am J Physiol 254: g254-g263; Lang and Marvig, 1989, Am J Physiol 256: g92-g99). Although apomorphine induces emesis by interaction with the D2-receptors in the chemoreceptor trigger zone, it also has an anti-emetic activity once it crosses the blood-brain barrier and interacts with the μ-receptors in the centrally located vomiting centre; this can lead to suppression of emesis and why if a therapeutic dose fails to induce emesis, a second dose is usually ineffective (Scherkl et al., 1990, J Vet Pharmacol Therap 13: 154-158).

Routes of administration for apomorphine and corresponding traditional dose forms which have been investigated and reported in dogs include parenteral, sublingual, nasal, rectal and ocular (Abdallah and Tye, 1967, Am J Dis Child 113: 571-575; Hackett, 2000, Clin Tech Small Anim Pract 15: 82-87; Harrison et al., 1972, J Am Vet Med Assoc 160: 85-86). For induction of emesis the most practical route has been parenteral and the drug is usually given at a dose of 0.08 mg/kg intravenously. A method of administration which avoids injection and yet allows a controlled rate of administration could reduce the major disadvantages of this drug which are toxicity and variability of response. If emesis were the therapeutic end point and drug absorption could be abruptly stopped at that point, some of the adverse effects related to overdosage might be avoided and the toxicity associated with the inherent interpatient variability seen with apomorphine might be reduced through the use of a controlled-release drug delivery system. Since the drug has been shown to be absorbed systemically in dogs after ocular administration (Abdallah and Tye, 1967; Harrison et al., 1972), incorporation of the drug into a polymeric matrix and application of this device to the eye could result in controlled release and absorption of the apomorphine. When the therapeutic end-point of emesis is reached, the device could be removed from the eye thus removing the drug reservoir and stopping further absorption of the drug. Since the clinician would want a prompt response to the drug, release of the drug from the polymer matrix would have to be complete in 5-10 minutes.

An insert meeting the above criteria was formulated using a gelatin-glycerin matrix and in-vitro testing of the inserts showed that at 34° C. which is the anticipated temperature of the conjunctival sac (Fink et al., 1988, Int J Clin Monitoring and Computing 5: 37-43; Efron et al., 1988, Curr Eye Res 7: 615-618), drug release was through both diffusion and erosion controlled mechanisms and an acceptable time window of 15 minutes was achieved. Stability testing demonstrated a shelf-life of at least one year and a method of fabrication resulting in the production of sterile inserts was established.

As part of the product evaluation, these inserts were made available to veterinary clinics interested in using and evaluating the inserts in canine patients. The dose of apomorphine recommended for induction of emesis using the ocular route was set at 0.1 mg/kg and since the weights of the patients could range from 1 to 100 kg, the drug load or amount of apomorphine in each insert was problematical. Although a series of inserts with different drug loads for different weight ranges might be appropriate, smaller patients would be put at risk if an insert containing an inappropriate load were used so only inserts carrying 2 mg of apomorphine were used for this trial.

The ocular inserts developed and described as above were used for this study. In the interest of patient safety, only one strength of insert was used and it carried a drug load of 2 mg of apomorphine; since the recommended emetic dose by the ocular route is to be 0.1 mg/kg and the anticipated patient weight range was from 1 to 100 kg, application of one insert would be appropriate for patients with weights ranging from 10 to 20 kg; smaller patients would require a portion of one insert and larger patients would require the application of multiple inserts.

In order to provide a basis of comparison and a control, a small series of patients were treated with apomorphine administered by the intravenous route. The drug was supplied as a single-use 2 mL vial containing 1 mg·mL−1 of apomorphine HCI. Sodium chloride was used as a tonicity adjuster, sodium metabisulfite 0.1% as an antioxidant and the solution was buffered to a pH of 5.5 using a phosphate buffer in water for injection.

Inserts were supplied to the participating clinics along with an information sheet and case report questionnaire. The information sheet described the inserts as to their use, dosage, storage and handling; the questionnaire supplied for each insert recorded the breed and weight of the patient, the reason for use and the nature of the intoxication, the time to emesis, dose applied and adverse effects noted, the ease of use and the usefulness of the product for this particular case. A list of potential adverse effects including prolonged vomiting, tachycardia, excitation, respiratory depression, bradycardia, sedation, ocular irritation and other were on the questionnaire and the clinician was asked to assign a grade for each. The grades were subjective and ranged from 0 to 5 with 5 being severe and 0 being not present. The ease of use and usefulness for each case was also assigned a subjective grade from 0 to 5 with 5 representing very easy to use and very useful.

Participants were instructed that the apomorphine ocular inserts were a unique experimental delivery system for use in inducing emesis in canine patients and that each disc contains 2 mg of apomorphine HCI in a non-irritating, biocompatible polymer.

Instructions stated that: The insert should be carefully removed from the protective wrapping by peeling the paper backing off of the plastic well. Using sterile blunt forceps, the insert is placed into the lower subconjunctival space. It is useful to wet the eye and/or the insert with saline or artificial tears for about 10 seconds prior to insertion as this additional moisture allows the insert to soften and conform to the shape of the eye more quickly. Release of the drug occurs as the polymer hydrates and drug release continues as the insert remains in the eye. Once the clinical effect of emesis is realized, the insert should be removed or flushed out without delay to avoid further absorption of the drug.

The recommended emetic dose is 0.1 mg/kg therefore some patients may require two inserts (one in each eye) while smaller patient may only require part of an insert. It is important that drug be administered as a single dose and the insert(s) removed promptly after emesis has occurred. Due to the toxic nature of the drug, the insert should be handled with care. Overdosage symptoms of respiratory depression can be treated with a narcotic antagonist such as naloxone, continuing emesis with metoclopramide and bradycardia may be treated with atropine. The purpose of this dose form is to allow drug absorption to occur at a controlled rate and once the therapeutic goal of emesis is achieved, to remove the drug reservoir, stop drug absorption and avoid overdosage. Some patients will be resistant to the emetic action of apomorphine and for these, oral hydrogen peroxide could be used rather than a second dose of apomorphine. This product should only be used in canine patients.

5001 reports for patients receiving apomorphine in the form of an ocular insert were available for analysis.

In the study population patient weights ranged from 1 to 80 kg with a median weight of 16.0 kg (25%=8.0 kg 75%=28.0 kg).

Emesis occurred in approximately 35% of the patients within 3-5 minutes or within 6-10 minutes for approximately 30% of the patients or within 11-15 for approximately 10% of the patients or within 1-2 minutes for approximately 5% of the patients. More than 15 minutes was considered to be a failure as discussed herein but for approximately 10% of the patients, emesis did occur after more than 15 minutes. For the remaining 10%, emesis did not occur. Thus, within 15 minutes after administration of the ocular insert, emesis occurred in approximately 80% (83.5%) of the patients and did not occur at all in 9.3% of the patients.

The data presented in Table 7 suggest that more therapeutic failures are associated with the heavier patients in the study population; the weights of the success group were compared with the weights of the failure group using a rank sum test (Mann-Whitney) and the weights between the two groups were found to be significantly different (p<0.001). This finding was somewhat unexpected since no difference was anticipated but this could be explained by the fact that the apomorphine release was time-dependent and if the release of drug was too slow, the arbitrary time of 15 minutes could pass before sufficient drug had been released

While not wishing to be bound to a specific hypothesis, it may be that since drug release from the insert is time dependent, with increasing weight more time will be required for sufficient drug to be absorbed and serum levels rise to those associated with emesis.

Apomorphine induces emesis by direct stimulation of the D2-receptors in the medullary chemoreceptor zone (Mitchelson, 1992, Drugs 43: 295-315) which is outside of the blood brain barrier (Keith et al., 1981, J Vet Pharmacol Therap 4: 315-316) but apomorphine is also rapidly distributed from the serum across the blood brain barrier and into the central nervous system where it is able to interact with the μ-opioid receptors (Scherkl et al., 1990, J Vet Pharmacol Therap 13: 154-158; Przedborski et al., 1995, Mov Discord 10: 28-36). Blancquqert et al clearly demonstrated that preceptor agonists in the central nervous system have an antemetic activity and this has been supported by other studies (Barnes et al., 1991, Neuropharmacology 30: 1073-1083; Bonuccelli et al., 1991, Clin Neuropharmacol 14: 442-449). The net effect of this is that apomorphine-induced emesis may be self-limiting and administration of the second half of the dose will not likely induce emesis.

Of the total sample population, 1382 patients had less than the recommended dose of 0.1 mg/kg applied and for these patients the median weight was 30 kg (25 and 35 kg for 25 and 75% respectively), the median time to emesis was 10 minutes (5 and 15 minutes for 25 and 75% respectively) and the median dose applied was 0.0769 mg/kg (0.0667 and 0.0857 mg/kg respectively). In this group, the success rate was 73% and the failure rate was 27%; about 15% of the patients experienced no emesis. Patients were grouped according to the dosage applied and the success/failure rate as well as the number of patients experiencing no emesis determined. These data are presented in Table 9.

A smaller group of patients received apomorphine by the intravenous route (n=32) at a dose of 0.03-0.04 mg kg-1. In this study population patient weights ranged from 1 to 50 kg with a median weight of 18.0 kg (11.0 and 27 kg for 25 and 75% respectively).

The weights for the small population treated with intravenous apomorphine were compared to those in the insert study population using a rank sum test (Mann-Whitney) and there was no significant difference in the weights between the two groups (p=0.446). The data from the group receiving intravenous apomorphine were further grouped as to success and failure with failure being considered as a time to emesis longer than 15 minutes. These data are presented in Table 10.

Overall, the time to emesis with the intravenous apomorphine was much shorter at a median of 1.0 minutes compared to the inserts where the overall time to emesis was approximately 6.0 minutes (p<0.001) and the success rate with the IV route was better at 90.6% as opposed to the ocular route where the overall success rate was 83.5%; although if only the patients where there is assurance that the apomorphine was given as a single dose i.e. with a body weight of 20 kg or less are considered, the success rate with the inserts increases to 87.1%. In terms of patients showing no emesis at all, the rate with the IV route was 9.4% and with the ocular route 9.3%.

The adverse effects reported included prolonged vomiting, tachycardia, excitation, respiratory depression, bradycardia, sedation and ocular irritation. On the report forms supplied with the product, if any of these occurred the clinicians were asked to grade them on a subjective scale of 1 to 5 with a score of 1 being mild and 5 being severe. The clinicians were also asked to rate the ease of use of the inserts for each case with a score of 5 being very easy to use and 1 being very difficult. The patients in whom adverse effects were noted were grouped according to the adverse effect and the mean weight, time to emesis and dose applied are summarized in Table 11. Since low-level ocular irritation occurred in almost every case, only scores of 2 or greater are reported for that category.

In the patient group receiving apomorphine by the intravenous route the adverse reactions seen are presented in Table 12.

Ocular irritation with the inserts was widely seen; virtually all of the reports indicated at least a low level of irritation as evidenced by inflammation and tearing but many of these rated the severity with a score of zero stating that it was not of clinical consequence. There are four possible explanations for the irritation seen; simple foreign body irritation is likely a factor (Acosta et al., 2001, Invest Opthalmol Vis Sci 42: 2063-2067); the excipients particularly the residual ascorbic acid and sodium bisulfite and the apomorphine itself may produce a hypertonic microenvironment and cause subsequent irritation (Fassihi and Naidoo, 1989, S Afr Med J 75: 233-235); mechanical trauma resulting from the placement of the insert may be a factor (Acosta et al., 2001) and lastly, there maybe some inherent irritation factor associated with apomorphine. A small amount of local irritation is desirable to ensure that there is sufficient tearing to allow swelling of the insert and subsequent drug release but the insert should not cause serious patient discomfort and certainly not cause tissue damage.

The frequency of ocular irritation was 16.2% with a severity scale value of 3 (25%=2, 75%=3) and no association of irritation with any particular group could be seen with the patients grouped by weight or time to emesis. The majority of reports indicated that ocular irritation was transient and resolved quickly after removal of the insert but there were 24 cases which were assigned a severity score of 5 and in 6 of these cases the reports indicated corticosteroid eye drops were administered to successfully resolve the inflammation.

Apomorphine itself appears to have some property causing tissue irritation but the etiology remains unknown. Dewey (Dewey et al., 1998, Mov Disord 3: 782-787) investigated the use of an apomorphine nasal spray to treat off-on fluctuations in Parkinson's disease patients and found although the treatment worked well, a very high incidence of severe nasal irritation was a major drawback to this mode of therapy

The frequency of adverse effects seen with the inserts and apomorphine given intravenously are summarized together in Table 13 and within the limits of this trial the inserts appeared to have been successful in reducing the overall frequency of adverse effects and specifically the tachycardia which would be associated with high serum levels of apomorphine.

Patients were also classified into groups based on the material ingested. The groupings were, in order of frequency, rodenticide, medication, chocolate, foreign object, unknown, dietary, slug bait, antifreeze, insecticide, plants and other. In the classification ‘Unknown’ the usual situation involved the patient presenting to the clinic with symptoms of intoxication but a causative agent could not be identified. The classification ‘Plants’ included ingestion of houseplants, ornamental garden flowers and bulbs, tobacco products, mushrooms/toadstools and marijuana. Most of the cases in ‘Dietary’ involved consumption of spoiled food, carrion, and compost but cases where the patient ate excessive amounts of pet or human food were also included into this classification. These cases are often referred to as dietary indiscretions. The group ‘Other’ was used for miscellaneous materials which did not fit into any of the categories; many of these cases involved the consumption of household chemicals particularly bone meal-based garden fertilizer and patients requiring pre-surgical emesis were also included in this group. The ‘Foreign object’ category included non-food items and most commonly included articles of clothing and toys such as tennis balls.

The patient population was arranged into groupings based on the nature of the intoxicant and the time to emesis profile, success/failure rate and percentage of patients showing no emesis were determined and these data are presented in Table 14.

While the preferred embodiments of the invention have been described above, it will be recognized and understood that various modifications may be made therein, and the appended claims are intended to cover all such modifications which may fall within the spirit and scope of the invention.

TABLE 1
Matrix formulations.

| | | |
|---|---|---|
| Formulation | Polymer | Plasticizer |

| | | | | |
|---|---|---|---|---|
| 1 | Gelatin | 4.75 g | Ethyl Citrate | 0.25 g |
| 2 | Gelatin | 4.50 g | Ethyl Citrate | 0.50 g |
| 3 | Gelatin | 4.00 g | Ethyl Citrate | 1.00 g |
| 4 | Gelatin | 3.50 g | Glycerin | 1.50 g |
| 5 | Gelatin | 3.00 g | Glycerin | 2.00 g |
| 6 | Gelatin | 2.50 g | Glycerin | 2.50 g |
| 7 | PVP | 4.75 g | Ethyl Citrate | 0.25 g |
| 8 | PVP | 4.50 g | Ethyl Citrate | 0.50 g |
| 9 | PVP | 4.00 g | Ethyl Citrate | 1.00 g |
| 10 | PVP | 3.50 g | Glycerin | 1.50 g |
| 11 | PVP | 3.00 g | Glycerin | 2.00 g |
| 12 | PVP | 2.50 g | Glycerin | 2.50 g |
| 13 | HPMC | 4.75 g | Ethyl Citrate | 0.25 g |
| 14 | HPMC | 4.50 g | Ethyl Citrate | 0.50 g |
| 15 | HPMC | 4.00 g | Ethyl Citrate | 1.00 g |
| 16 | HPMC | 3.50 g | Glycerin | 1.50 g |
| 17 | HPMC | 3.00 g | Glycerin | 2.00 g |
| 18 | HPMC | 2.50 g | Glycerin | 2.50 g |

TABLE 2
Attribute scores for matrix formulations

| Formulation | Flexibility | Clarity | Tackiness | Integrity | Total |
|---|---|---|---|---|---|
| 1 | 1 | 1 | 5 | 4 | 11 |
| 2 | 1 | 1 | 5 | 4 | 11 |
| 3 | 1 | 1 | 5 | 4 | 11 |
| 4 | 4 | 3 | 5 | 5 | 17 |
| 5 | 5 | 3 | 5 | 6 | 19 |
| 6 | 5 | 3 | 4 | 5 | 17 |
| 7 | 1 | 1 | 4 | 3 | 9 |
| 8 | 1 | 1 | 4 | 3 | 9 |
| 9 | 1 | 1 | 4 | 3 | 9 |
| 10 | 5 | 3 | 2 | 4 | 14 |
| 11 | 5 | 3 | 2 | 4 | 14 |
| 12 | 5 | 3 | 2 | 4 | 14 |
| 13 | 4 | 3 | 4 | 4 | 15 |
| 14 | 3 | 2 | 4 | 4 | 13 |
| 15 | 2 | 1 | 4 | 4 | 11 |
| 16 | 3 | 2 | 5 | 4 | 14 |
| 17 | 4 | 2 | 5 | 4 | 15 |
| 18 | 5 | 2 | 5 | 3 | 15 |

TABLE 3
summary of factorial design

| | | VALUE | | |
|---|---|---|---|---|
| Factor | Code | +1 | 0 | −1 |
| Polymer | b1 | Gelatin | PVP | HPMC |
| Plasticizer | b2 | Ethyl Citrate | | Glycerin |
| Plasticizer level | b3 | High | Intermediate | Low |
| Response score | y | | | |

TABLE 4
Results of MLR of coded formulation factors

| Factor | Code | Coefficient | p |
|---|---|---|---|
| Polymer | b1 | 0.250 | 0.637 |
| Plasticizer | b2 | −2.222 | <0.001 |
| Plasticizer level | b3 | 0.250 | 0.637 |
| Polymer/plasticizer | b12 | −1.250 | 0.035 |
| Polymer/plasticizer level | b13 | −0.375 | 0.564 |
| Plasticizer/plasticizer level | b23 | 0.417 | 0.436 |
| Polymer/plasticizer/plasticizer level | b123 | −0.625 | 0.344 |

TABLE 5
Formulations Evaluated

| Component | Quantity (g) | A | B | C | D |
|---|---|---|---|---|---|
| Apomorphine | 0.460 | + | + | + | + |
| Gelatin | 2.85 | + | + | + | + |
| Glycerin | 1.95 | + | + | + | + |
| Sodium metabisulfite | 0.050 | + | − | + | − |
| Ascorbic Acid | 0.050 | + | + | − | − |

TABLE 6
Percent of apomorphine remaining after storage
at 80° C. for formulations A to D

| Day | A | B | C | D |
|---|---|---|---|---|
| 0 | 99.24 ± 1.882 | 100.5 ± 2.592 | 91.72 ± 1.376 | 103.0 ± 2.881 |
| 3 | 84.92 ± 1.070 | 89.24 ± 0.417 | 88.19 ± 1.079 | 92.29 ± 5.015 |
| 6 | 88.49 ± 0.441 | 87.84 ± 1.111 | 76.38 ± 1.179 | 105.2 ± 5.796 |
| 9 | 83.78 ± 1.960 | 100.4 ± 1.056 | 75.97 ± 0.809 | 85.79 ± 3.891 |
| 12 | 74.72 ± 1.942 | 81.31 ± 1.012 | 71.91 ± 0.827 | 78.94 ± 6.288 |
| 15 | 67.36 ± 1.203 | 88.68 ± 0.919 | 78.10 ± 0.959 | 84.09 ± 3.890 |
| 18 | 71.33 ± 1.068 | 73.00 ± 1.978 | 69.80 ± 0.677 | 78.41 ± 3.514 |
| 21 | 71.03 ± 2.099 | 80.24 ± 1.226 | 50.91 ± 0.644 | 70.36 ± 3.487 |
| 24 | 62.28 ± 1.437 | 71.90 ± 1.019 | 57.29 ± 1.089 | 74.97 ± 4.241 |
| 27 | 63.22 ± 1.209 | 85.61 ± 1.470 | 51.51 ± 1.501 | 68.07 ± 2.991 |
| 30 | 57.14 ± 1.483 | 64.98 ± 0.783 | 44.04 ± 1.057 | 58.81 ± 2.816 |
| 33 | 63.29 ± 1.668 | 63.54 ± 0.477 | 56.50 ± 1.717 | 66.02 ± 2.935 |
| 36 | 61.64 ± 0.847 | 61.15 ± 0.830 | 53.62 ± 1.667 | 60.23 ± 2.364 |
| 39 | 49.33 ± 1.371 | 60.55 ± 1.341 | 34.22 ± .0774 | 55.28 ± 2.783 |

Values: mean ± std dev n = 4

TABLE 7
Comparison of apomorphine insert success and failure rates

| | Success | Failure | Difference |
|---|---|---|---|
| Time to emesis (min) | 6.0 | — | — |
| Median Weight (kg) | 15 | 25 | p < 0.001 |
| % of Patients | 83.5 | 16.5 | — |
| n | 4174 | 827 | — |

Values: Mean ± std deviation

Table 8 Patients categorized by weight and groups compared as to time to emesis, failure and no emesis

| | Time to Emesis - Cumulative % of Patients | | | | | | |
|---|---|---|---|---|---|---|---|
| Wt | 0-2 | 3-5 | 6-10 | 11-15 | | No | |
| (kg) | min | min | min | min | Failure | Emesis | n |
| 1-5 | 13.2 | 70.9 | 91.1 | 94.7 | 5.3 | 3.2 | 660 |
| 6-10 | 6.8 | 55.3 | 84.3 | 92.2 | 7.8 | 4.2 | 1123 |
| 11-15 | 3.5 | 40.8 | 76.0 | 85.0 | 15.0 | 9.3 | 633 |
| 16-20 | 2.7 | 37.8 | 68.6 | 82.0 | 18.0 | 11.2 | 590 |
| 21-25 | 2.3 | 25.0 | 57.5 | 75.8 | 24.2 | 13.9 | 569 |
| 26-30 | 2.2 | 22.9 | 57.0 | 73.7 | 26.3 | 13.8 | 558 |
| 31-35 | 1.4 | 24.2 | 60.5 | 77.0 | 23.0 | 12.7 | 418 |
| 36-40 | 3.5 | 22.3 | 59.7 | 76.3 | 23.7 | 11.3 | 283 |
| >40 | 0 | 15.6 | 44.3 | 66.5 | 33.5 | 18.6 | 167 |

TABLE 9

| Dose Applied | | | | |
|---|---|---|---|---|
| (mg · kg⁻¹) | Success | Failure | No Emesis | n |
| 0.36-0.40 | 573 (93.5%) | 40 (6.5%) | 24 (3.9%) | 613 |
| 0.31-0.35 | 199 (93.4%) | 14 (6.6%) | 10 (4.7%) | 213 |
| 0.26-0.30 | 200 (88.5%) | 26 (11.5%) | 17 (7.5%) | 226 |
| 0.21-0.25 | 361 (91.2%) | 35 (8.8%) | 22 (5.6%) | 396 |
| 0.16-0.20 | 605 (87.1%) | 90 (12.9%) | 42 (6.0%) | 695 |
| 0.11-0.15 | 826 (83.4%) | 164 (16.6%) | 91 (9.2%) | 990 |
| 0.06-0.10 | 1267 (76.1%) | 399 (23.9%) | 231 (13.9%) | 1666 |
| <0.06 | 143 (70.8%) | 59 (29.2%) | 28 (13.9%) | 202 |

TABLE 10

| | Success | Failure | Difference |
|---|---|---|---|
| Time to emesis (min) | 1.0 | — | — |
| Median Weight (kg) | 16 | 21 | p = 0.943 |
| % of Patients | 90.6 | 9.4 | — |
| n | 29 | 3 | — |

Values: Mean ± std deviation

TABLE 11

| Category | Frequency (%) | Score |
|---|---|---|
| Prolonged vomiting | 2.3 | 2 (1-3) |
| Tachycardia | 0.6 | 2 (1-2) |
| Excitation | 0.4 | 2 (1-2) |
| Respiratory | 0.6 | 2 (1-2) |
| depression | | |
| Bradycardia | 0.7 | 2 (1-3) |
| Sedation | 11.1 | 2 (1-3) |
| Ocular irritation | 16.2 | 3 (2-3) |
| Difficulty with use | 6.3 | 3 (3-4 |

Median (25%-75%) n = 5001

TABLE 12

| Category | Frequency (%) | Score |
|---|---|---|
| Prolonged vomiting | 3.1 | 3 (3-3) |
| Tachycardia | 15.6 | 2 (2-3) |
| Excitation | — | — |
| Respiratory | — | — |
| depression | | |
| Bradycardia | — | — |
| Sedation | 43.8 | 2 (1-2) |

Median (25%-75%) n = 32

TABLE 13

% Frequency

| Adverse effect | Ocular | IV |
|---|---|---|
| Prolonged vomiting | 2.3 | 3.1 |
| Tachycardia | 0.6 | 15.6 |
| Excitation | 0.4 | — |
| Respiratory | 0.6 | — |
| depression | | |
| Bradycardia | 0.7 | — |
| Sedation | 11.1 | 43.8 |
| n | 5001 | 32 |

TABLE 14

| | Time to emesis - % of Patients (Cumulative) | | | | | | |
|---|---|---|---|---|---|---|---|
| | 1-2 | 3-5 | 6-10 | 11-15 | | No | |
| Toxin | min | min | min | min | Failure | Emesis | n |
| Rodenticide | 4.04 | 41.6 | 71.5 | 85.6 | 14.4 | 6.30 | 1461 |
| Medication | 6.73 | 45.5 | 75.8 | 86.2 | 13.8 | 7.10 | 803 |
| Chocolate | 6.45 | 46.5 | 76.3 | 86.7 | 13.4 | 6.89 | 682 |
| Foreign object | 2.85 | 34.9 | 69.6 | 82.6 | 17.4 | 8.39 | 596 |
| Unknown | 6.37 | 35.7 | 71.5 | 79.3 | 20.7 | 15.6 | 487 |
| Dietary | 2.33 | 35.0 | 64.7 | 81.0 | 19.0 | 11.3 | 300 |
| indiscretion | | | | | | | |
| Slug bait | 3.98 | 30.7 | 53.4 | 63.1 | 36.9 | 30.1 | 176 |
| Antifreeze | 4.83 | 42.8 | 69.7 | 84.8 | 15.2 | 11.7 | 145 |
| Insecticide | 6.62 | 39.7 | 76.5 | 86.0 | 14.0 | 7.35 | 136 |
| Plant | 3.85 | 48.5 | 75.4 | 82.3 | 17.7 | 8.46 | 130 |
| Other | 2.35 | 24.7 | 56.5 | 72.9 | 27.1 | 21.2 | 85 |
| All | 4.84 | 40.6 | 71.5 | 83.5 | 16.5 | 9.30 | 5001 |

1. A removable ocular insert comprising an effective amount of apomorphine in a polymer matrix.

2. The ocular insert according to claim 1 wherein the apomorphine is apomorphine HCI.

3. The ocular insert according to claim 1 wherein the polymer matrix is a mixture of glycerin and gelatin.

4. The ocular insert according to claim 3 wherein the glycerin is mixed with the gelatin at between a 1:1 to 1:1.5 ratio.

5. A method of inducing emesis in an animal in need of such treatment comprising inserting into the eye of said animal a removable ocular insert comprising an effective amount of apomorphine in a polymer matrix, and once emesis has occurred, removing the insert from the eye of said animal.

6. The method according to claim 5 wherein the apomorphine is apomorphine HCI.

7. The method according to claim 5 wherein the polymer matrix is a mixture of glycerin and gelatin.

8. The method according to claim 7 wherein the glycerin is mixed with the gelatin at between a 1:1 to 1:1.5 ratio.
